(19) United States
(12) Patent Application Publication
(10) Pub. No.: US 2007/0106282 A1
(43) Pub. Date: May 10, 2007

(54) Determination of the position of an anatomical element

(75) Inventor: Stephane Lavallee, Saint Martin D'Uriage (FR)

Correspondence Address: PLEVY & HOWARD & DARCY P.C., P.O. BOX 226, Fort Washington, PA, 19034, US

(73) Assignee: PERCEPTION RAISONNEMENT ACTION EN MEDECINE, 38700 LA TRONCHE (FR)

(21) Appl. No.: 10/554,981

(22) PCT Filed: Apr. 30, 2004
(86) PCT No.: PCT/FR04/50180

§ 371 Date: Dec. 4, 2006

(30) Foreign Application Priority Data
May 2, 2003 (FR) 03/05416

(51) Int. Cl. A61B 17/00

(52) U.S. Cl. 606/1

(57) ABSTRACT

The invention relates to a method for determining the position of an anatomical element (12) of a patient (10), consisting in determining at an initial moment the position of the anatomical element from positions of anatomical points (14, 16, 18) which are characteristic of said patient; disposing a marking device (30) on the skin of the patient, said marking device being fixed in relation to the anatomical element; determining the position of the marking device, whereby the anatomical element is substantially the position determined at the initial instant; determining the relative position of the anatomical element in relation to the marking device; covering the marking device at least partially with at least one operational field; and determining the position of the marking device covered at least partially with said operational field and deducing the position of the anatomical element.

The present invention relates to a method and a device for determining the position of an anatomic element.

In a computer-assisted surgical operation, it may be necessary to determine the position of a patient's anatomic element with respect to a reference coordinate system. As an example, in the case of a computer-assisted arthroplasty of the hip, it is necessary to be able to continuously express, in the reference coordinate system, the equations of characteristic planes associated with the patient's pelvis.

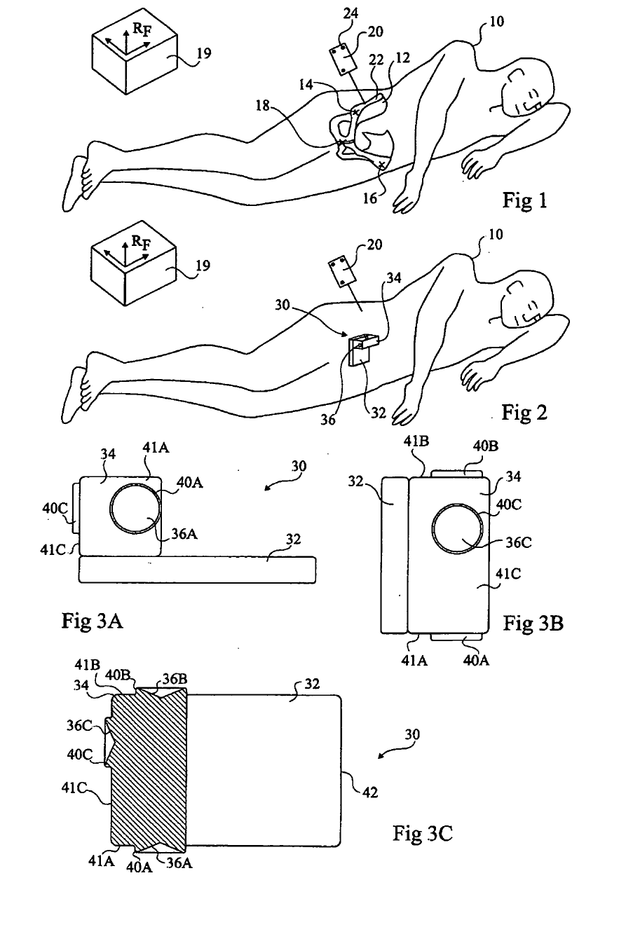

FIG. 1 schematically shows the body of a patient 10 placed in lateral decubitus position for a computer-assisted hip arthroplasty operation. Pelvis 12 of patient 10 has been shown. At a given time, the position of pelvis 12 can be defined from the positions of the three following characteristic anatomic points, represented by crosses: the two anteroposterior iliac spines 14, 16, and the pubic symphysis 18. The positions of anatomic points 14, 16, 18 are determined, at an initial time, by means of a probe (not shown) having its position determined by a location system 19. The system may be an optical, magnetic, ultrasound, optical fiber, etc. location system. A computer (not shown), connected to location system 19, is capable of determining the position of pelvis 12 from the position of the probe in a reference coordinate system R_{F} associated with location system 19.

During the operation, it is necessary to continuously track the position of pelvis 12. For this purpose, a rigid body 20 is attached to pelvis 12, for example, on iliac crest 22. The location system continuously provides the position of rigid body 20 in reference coordinate system R_{F}. For this purpose, location system 19 comprises, for example, means (not shown) for emitting an infrared radiation which is reflected by reflective portions 22 of rigid body 20, the reflected radiation being sensed by cameras (not shown) sized together and supported by location system 19. Pelvis 12 being attached with respect to rigid body 20, the position of pelvis 12 can be deduced in reference coordinate system RF throughout the surgical operation based on the position of rigid body 20 in reference coordinate system R_{F}.

To obtain the relative position between pelvis 12 and rigid body 20, it is necessary to determine the positions of pelvis 12 and of rigid body 20 at very close times to avoid a disturbance of the measurements due to the displacements of patient 10 or of location system 19. For this purpose, the position of pelvis 12 in reference coordinate system R_{F} can be determined according to the following steps:

attaching rigid body 20 on iliac crest 22 of pelvis 12 of patient 10 placed in lateral decubitus position and determining the position of rigid body 20 in reference coordinate system R_{F};

determining positions of the three characteristic anatomic points 14, 16, 18 in reference coordinate system R_{F} and determining the relative position between pelvis 12 and rigid body 20;

removing rigid body 20;

preparing the patient for the operation, especially arranging the operating drapes (not shown) to define the operation area;

reproducibly placing back rigid body 20;

continuously determining the position of rigid body 20, and accordingly, the position of pelvis 12 in reference coordinate system R_{F}.

A disadvantage of such a method is that rigid body 20 is arranged on pelvis 12 to determine the relative position between pelvis 12 and rigid body 20 before the preparation of patient 10. This provides an additional risk of infection of patient 10. Further, rigid body 20 being temporarily removed to prepare patient 10, it is difficult to place it back on iliac crest 22 exactly at the position that it had before the preparation of patient 10.

Another conventional method for determining the position of pelvis 12 in reference coordinate system R_{F} comprises the steps of:

preparing patient 10, especially placing patient 10 in lateral decubitus position, and arranging the operating drapes;

performing an incision to have access to the area to be operated on;

arranging rigid body 20 on pelvis 12 through the incision previously performed and continuously determining the position of rigid body 20 in reference coordinate system R_{F}; and

palpating the three characteristic anatomic points 14, 16, 18 and determining the relative position between pelvis 12 and rigid body 20.

A disadvantage of such a method is that the palpating of characteristic anatomic points 14, 16, 18 is very delicate since the latter are then of difficult access, especially due to the operating drapes covering the body of patient 10.

The present invention provides a device for determining the position of a patient's anatomic element which does not exhibit the above-mentioned disadvantages.

The present invention also provides a method for determining the position of a patient's anatomic element that can be implemented prior to the patient's surgical operation and in the presence of the operating drapes.

For this purpose, it provides a tracking device intended to bear against the skin of a patient in a computer-assisted surgical operation, comprising at least three non-aligned marks intended to be palpated through at least one operating drape and each defining a guiding point.

According to another aspect of the invention, at least one mark is a conical recess surrounded with a protruding ring-shaped edge.

According to another aspect of the invention, the device comprises at least two distinct mechanical parts intended to bear against the patient's skin, each distinct mechanical part comprising at least one mark.

According to another aspect of the invention, the device comprises a parallelepipedal block, the three marks being arranged on three distinct surfaces of the block.

According to another aspect of the invention, two marks are arranged on two parallel surfaces, symmetrically with respect to a plane equidistant to the two parallel surfaces, the third mark being arranged on a surface perpendicular to the two parallel surfaces, closer to one of the parallel surfaces.

The present invention also provides a method for determining the position of a patient's anatomic element comprising the steps of determining, at an initial time, the position of the anatomic element from the positions of characteristic anatomic points of the patient; arranging, on the patient's skin, a tracking device fixed with respect to the anatomic element; determining the position of the tracking device, the anatomic element being substantially at the position determined at the initial time; determining the relative position of the anatomic element with respect to the tracking device; at least partially covering the tracking device with at least one operating drape; and determining the position of the tracking device at least partially covered with said at least one operating drape and deducing therefrom the position of the anatomic element.

According to another aspect of the invention, the positions of characteristic anatomic points of the patient are obtained by palpation of at least three anatomic points of the patient.

According to another aspect of the invention, the positions of characteristic anatomic points of the patient are obtained from ultrasound scan images of the patient.

According to another aspect of the invention, the positions of the tracking device are obtained by palpation of marks of the tracking device.

According to another aspect of the invention, the marks of the tracking device are palpated to determine the position of the tracking device in the absence of said at least one operating drape by means of a first probe comprising a spherical end having a first radius and the marks of the tracking device are palpated to determine the position of the tracking device at least partially covered with said at least one operating drape by means of a second probe comprising a spherical end having a second radius smaller than the first radius.

According to another aspect of the invention, the marks are conical recesses.

According to another aspect of the invention, the positions of the marks of the tracking device determined in the absence of said at least one operating drape are displayed on a display screen to ease the palpation of the marks of the tracking device to determine the position of the tracking device at least partially covered with said at least one operating drape.

According to another aspect of the invention, the displayed positions of the marks of the tracking device determined in the absence of said at least one operating drape are corrected based on the determined position of a first mark of the tracking device at least partially covered with said at least one operating drape.

The foregoing object, features, and advantages of the present invention, as well as others, will be discussed in detail in the following non-limiting description of specific embodiments in connection with the accompanying drawings, among which:

FIG. 1, previously described, schematically illustrates a conventional method for determining the position of a patient's anatomic element;

FIG. 2 schematically illustrates an example of a method according to the invention for determining the position of the patient's anatomic element;

FIGS. 3A to 3C show views of an example of the forming of a tracking device according to the present invention;

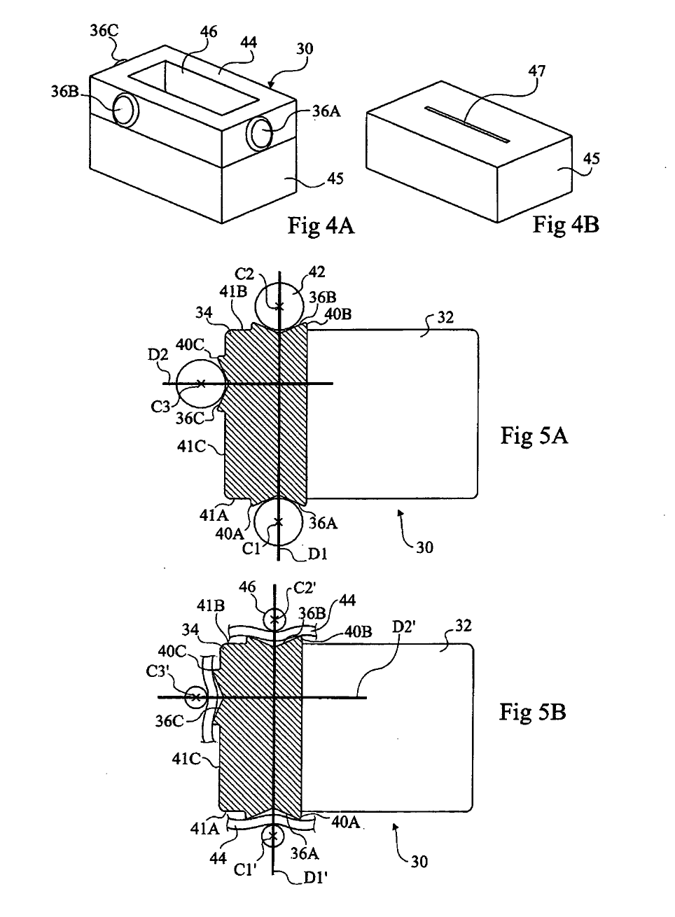

FIG. 4A is a perspective view of a variation of the tracking device of FIGS. 3A to 3C;

FIG. 4B is a perspective view of an element of the device of FIG. 4A;

FIGS. 5A and 5B show in further detail two steps of the method illustrated in FIG. 2;

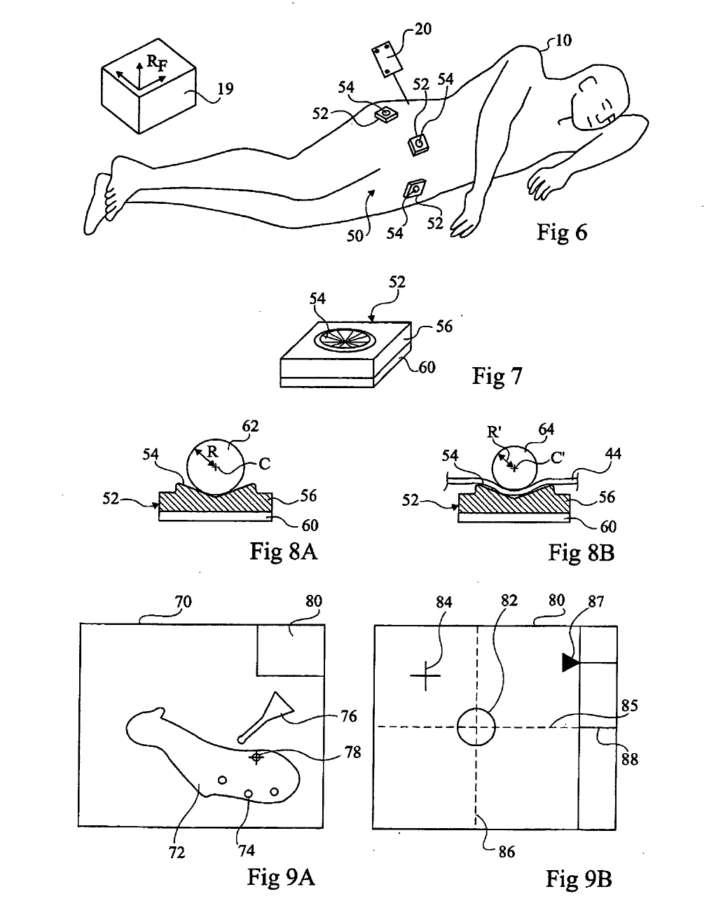

FIG. 6 schematically illustrates another example of the method according to the invention;

FIG. 7 is a perspective view of another example of the forming of the device according to the invention;

FIGS. 8A and 8B show in further detail two steps of the method illustrated in FIG. 6; and

FIGS. 9A and 9B show information displayed on a display screen on implementation of the method according to the present invention.

In the different drawings, same elements are designated with same reference numerals.

FIG. 2 shows the body of patient 10 arranged in lateral decubitus position on an operation table (not shown). A tracking device 30 is applied on the skin of patient 10. According to the present example of embodiment, device 30 comprises a base 32 and a block 34 attached to base 32. Block 34 comprises at least three marks 36A, 36C. By means of a probe with a spherical tip (not shown), it is possible to obtain the position of tracking device 30 in reference coordinate system R_{F} from the palpation of marks 36A, 36C. As will described in further detail hereafter, the palpation of marks 36A, 36C of tracking device 30 can be performed even when the latter is covered with operating drapes. Tracking device 30 is attached to patient 10 by applying base 32 on the patient's pubis. Tracking device 30 is then substantially fixed with respect to pelvis 12.

An example of a method according to the invention for determining the position of pelvis 12 comprises the steps of:

arranging on patient 10 tracking device 30 according to the present invention;

determining the position of the tracking device with respect to reference coordinate system R_{F};

determining the position at an initial time of pelvis 12 in reference coordinate system R_{F}, for example by palpation of characteristic anatomic points of patient 10;

determining the relative position between pelvis 12 and tracking device 30;

preparing patient 10 for the operation, especially arranging the operating drapes (not shown) to define the sterile operation area;

arranging rigid body 20 on pelvis 12 of patient 10 and continuously determining the position of rigid body 12 in reference coordinate system R_{F};

determining again the position of tracking device 30 through the operating drapes in reference coordinate system R_{F}, and determining the relative position between tracking device 30 and rigid body 20; and

continuously determining the position of pelvis 12 in reference coordinate system R_{F}.

FIGS. 3A, 3B, and 3C respectively are a side view, a front view, and a top cross-section view of tracking device 30 of FIG. 2. Base 32 substantially has the shape of a parallelepipedal plate. Block 34, substantially parallelepipedal, is arranged along a side of base 32. On three surfaces, block 34 comprises a mark 36A, 36B, 36C in the form of a conical recess. Each conical mark 36A, 36B, 36C is surrounded with a ring-shaped rounded edge 40A, 40B, 40C protruding with respect to the corresponding surface 41A, 41B, 41C of block 34. As an example, the angles at the top of conical marks 36A, 36B, 36C are identical and equal to approximately 120° and rounded edges 40A, 40B, 40C have a diameter of approximately 20 mm and a height of approximately 3 mm. Two conical marks 36A, 36B are formed on two opposite parallel surfaces 41A, 41B of block 34. The third conical mark 36C is formed on a surface 41C of block 34 perpendicular to surfaces 41A, 41B and is closer to one of the two conical marks 36A, 36B.

Side 42 of base 32 may be aligned along the pubic symphysis 18 of patient 10 to ease the palpation of the pubic symphysis on palpation of the characteristic anatomic points of patient 10 to obtain the position of pelvis 12 in reference coordinate system R_{F} at the initial time. According to a variation, base 32 is not aligned with the pubic symphysis and comprises an opening through which the pubic symphysis is palpated on determination of the position of pelvis 12 at the initial time.

Base 32 may be applied against the patient's pubis via a device usually used to maintain patient 10 on the operation table in lateral decubitus position. According to a variation of the invention, tracking device 30 is directly integrated to the device maintaining the patient in lateral decubitus position. According to another variation, the tracking device is attached to the patient's body by an adhesive film.

FIG. 4A is a perspective view of a variation of tracking device 30 which is particularly advantageous when the device is integrated to a device maintaining the patient in lateral decubitus position. Tracking device 30 comprises a rigid frame 44, connected to the holding device, and on which are formed conical marks 36A, 36B, 36C. A foam block 45 is arranged on frame 44 and is compressed against the pubis of patient 10 by the holding device. Foam block 45 enables uniform distribution on the pubis of the forces exerted by the holding device. Frame 44 defines an opening 46 enabling the surgeon to have access to foam block 45.

FIG. 4B is a perspective view of foam block 45. Foam block 45 is crossed by a slot 47 to which the surgeon has access through opening 46 of tracking device 30. With an adapted tool, the surgeon can space apart the walls of slot 47 to clear a pathway to the pubis. The surgeon can then palpate the pubis, through the pathway thus cleared, to determine the position of the pubic symphysis, either directly with a probe, or with an echographic probe located in reference coordinate system R_{F}.

FIG. 5A illustrates a step of the method for determining the position of tracking device 30 illustrated in FIG. 2. A probe, having a single end 42 shown by a sphere in FIG. 5A, is placed successively at the level of each conical mark 36A, 36B, 36C. The probe is equipped with a rigid body, similar to rigid body 20 shown in FIG. 2, so that location system 19 determines the probe position and accordingly the position of the center of spherical end 42 of the probe in reference coordinate system R_{F}. Points C1, C2, C3, represented by crosses in FIG. 5A, correspond to the positions taken by the center of spherical end 42 of the probe when it is successively placed in marks 36A, 36B, 36C. The two conical marks 36A, 36B are arranged symmetrically with respect to each other so that points C1 and C2 define a straight line D1 perpendicular to surfaces 41A and 41B. Point C3 projects on straight line D1 at a point H and the straight line connecting C1 to H defines a straight line D2 perpendicular to D1. Straight lines D1 and D2 altogether completely define a three-dimensional reference frame characteristic of the position of the tracking device. Thus, from lines D1 and D2, the computer connected to location system 19 defines the position of the tracking device in reference coordinate system R_{F}.

To determine straight lines D1, D2, the computer may number points C1, C2, C3. To avoid ambiguities in the numbering, conical mark 36C is not placed at an equal distance from conical marks 36A, 36B but is closer, for example, to conical mark 36B.

FIG. 5B illustrates a subsequent step of the method for determining the position of tracking device 30 when operating drapes 44 cover patient 10, and especially tracking device 30. A probe comprising a spherical end 46, shown by a sphere in FIG. 5B, is then used. The recessed conical shape of marks 36A, 36B, 36C and the protruding shape of rounded edges 40A, 40B, 40C define characteristic surfaces which enable the surgeon to easily identify when the probe is in a proper position on the axis of the cone of a conical mark 36A, 36B, 36C. Indeed, by sliding the probe sphere along the cone walls as deep as possible, the sphere center is on the cone axis.

The conical shape of marks 36A, 36B, 36C and the presence of edges 40A, 40B, 40C enables, despite the presence of operating drapes 44, defining points C1′, C2′, C3′, represented by crosses in FIG. 5B, occupied by the center of spherical end 46 of the probe when it is placed at the level of conical marks 36A, 36B, 36C. Conical marks 36A, 36B being arranged on opposite surfaces 41A, 41B symmetrically with respect to each other, straight line D1′ defined by the two points C1′, C2′ is identical to straight line D1 of FIG. 5A. Similarly, the symmetry of revolution of mark 36C ensures that straight line D2′, defined from point C2′ and from straight line D1′, is identical to straight line D2. The computer thus determines a same position of the tracking device in reference coordinate system R_{F} as that obtained at FIG. 5A if the tracking device has not moved. In the present example of embodiment, spherical end 46 is shown, as an example, with a radius smaller than the radius of spherical end 42 of the probe of FIG. 5A. An advantage of tracking device 30 according to the present example of embodiment is that it enables correct determination of the position of tracking device 30 independently from the radius of the end of probe and from the thickness of operating drapes 44.

According to a variation of the present invention, instead of conical marks 36A, 36B, 36C, marks formed of raised areas which project out of surfaces 41A, 41B, 41C of tracking device 30, are provided. These are for example, pyramidal, conical, tapered, etc. surfaces distributed on block 34 and having their position determined by a probe of complementary shape. As compared with the previously-described tracking device, this amounts to inverting the geometrical shapes of the mark and of the probe.

According to a variation of the present invention, block 34 comprises more than three conical marks so that at least three conical marks are continuously accessible to the probe.

FIG. 6 illustrates another example of the method according to the invention for determining the position of pelvis 12 in reference coordinate system R_{F}. According to this example, a tracking device 50 formed of an assembly of markers 52, three markers being shown in FIG. 5 is provided. Each marker comprises a conical mark 54 which substantially has the same shape as conical marks 36A, 36B, 36C of device 30 of FIGS. 3A to 3C. Tracking device 50 comprises at least three markers 52 and preferably from five to six markers. Markers 52 are distributed on the patient at locations such that the relative positions between the markers remain unchanged even if patient 10 makes slight motions. Similarly to what has been previously described, the relative position between tracking device 50 and pelvis 12 is determined from the palpation of markers 52. After preparing patient 10 and arranging rigid body 20, the position of tracking device 50 is determined in reference coordinate system R_{F}, by palpation of markers 52 in the presence of the operating drapes covering patient 10. The relative position of tracking device 50 with respect to rigid body 12, and thus the relative position of pelvis 12 with respect to rigid body 20 can then be determined, and the position of pelvis 12 can then be determined in reference coordinate system R_{F}.

FIG. 7 shows a perspective view of an example of the forming of a marker 52 of FIG. 6. Marker 52 comprises a parallelepipedal or circular base 56. Conical mark 54 is formed at the center of a surface of the base. As for tracking device 30 of FIGS. 3A to 3C, a ring-shaped border 58 surrounding conical mark 54 protrudes from base 56 and eases the palpation of a point located on the axis of the cone of mark 54 when marker 52 is covered with an operating drape. Base 56 comprises an adhesive film 60 on the surface opposite to conical mark 54 for attaching marker 52 to patient 10, similarly to a cardiology electrode. According to a variation of the present invention, a mark formed of a raised area which protrudes from base 56 is provided instead of conical mark 54. It may for example be pyramidal, conical, tapered, etc. surfaces.

FIG. 8A illustrates a step of the method for determining the position of tracking device 50 illustrated in FIG. 6. A probe, a single end 62 of which is represented by a sphere of radius R in FIG. 8A, is successively placed on the axis of the cone of mark 54 of each marker 52. The recessed conical shape of mark 54 and the protruding shape of the rounded edge surrounding mark 54 define characteristic surfaces which enable the surgeon to easily identify when the probe is in a correct position at the level of conical mark 54. Location system 19 is capable of determining the position of center C of spherical end 62 placed in conical mark 54. Based on the positions of the centers determined for all markers 52, the computer determines the position of tracking device 50 in reference coordinate system R_{F}.

FIG. 8B illustrates a subsequent step of the method for determining the position of tracking device 30 when operating drapes 44 cover patient 10 and, more specifically, markers 52. A probe comprising a spherical end 64, represented in FIG. 8B by a sphere of radius R′ smaller than R, is then used. The difference between radiuses R and R′ substantially corresponds to the thickness of operating drape 44 covering marker 52 so that the position of center C′ of spherical end 64 with respect to base 56 is identical to the position determined for center C of spherical end 62 with respect to base 56 in FIG. 8A. Such a thickness may be empirically determined and be memorized by the computer. The thickness may also correspond to a parameter provided to the computer before preparation of patient 10 and which depends on the type of operating drape and of probe used. Based on the positions of the centers determined for all markers 52, the computer determines the position of tracking device 50 in reference coordinate system R_{F}.

According to another example (not shown) of embodiment of the tracking device according to the present invention, the device is formed of two bars. At the end of each bar are provided conical marks similar to previously-described conical marks 36A, 36B, 36C, 54. The two bars are placed independently from each other on the patient. Similarly to what has been previously described for tracking device 30, the computer determines a straight line for each pair of symmetrical conical marks of a bar. The computer also determines for each pair of conical marks of a bar a midpoint equidistant from the conical marks. An advantage of such a tracking device is that for each bar, the determination of the straight line and of the midpoint does not depend on the presence or on the absence of the operating drapes and thus on their possible thickness. Based on the two straight lines and on the two midpoints, the computer can then determine the position of the tracking device in reference coordinate system R_{F}.

According to another example (not shown) of embodiment of the tracking device according to the present invention, the tracking device comprises a cylindrical block of axis D, possibly hollow, comprising a cylindrical outer wall on which are distributed three marks. An advantage of such a tracking device is that the determination of axis D and of the plane comprising the three tracking points associated with the marks does not depend on the presence or on the absence of operating drapes, and thus on the thickness of such operating drapes. From the determination of axis D and of the plane associated with the marks, the computer can then determine the position of the tracking device in reference coordinate system R_{F}.

More generally, the tracking device comprises a lateral wall on which are distributed at least three marks. The shape of the marks is such that the guiding points obtained by palpation of the marks in the presence or in the absence of the operating drapes are in a same plane and that at least one point determined from the tracking points is invariant when it is determined in the presence or in the absence of operating drapes. For the tracking device shown in FIGS. 5A and 5B, such an invariant point is for example the point corresponding to the projection of C3 (respectively, C3′) on the straight line joining points C1 and C2 (respectively C1′ and C2′). For a cylindrical tracking device, an invariant point is for example the point equidistant to the guiding points.

In the previously-described examples of embodiment, a probe comprising a spherical end has been used. However, an intermediary pliers-shaped element may be used for the palpation of the tracking device. The pliers-shaped element comprises legs that can cooperate with the marks of the tracking device to ensure the attachment of the pliers-shaped element on the tracking device. The pliers-shaped element can be attached to the tracking device in the absence of operating drapes or with one or several operating drapes interposed between the tracking device and the pliers-shaped element. Such a pliers-shaped element itself comprises marks intended to be palpated, for example, by a probe comprising a spherical end. The pliers-shaped device is designed so that the marks of the pliers-shaped element are at the same relative positions with respect to the tracking device when the pliers-shaped element is attached to the tracking device in the presence or in the absence of operating drapes. The position of the tracking device in reference coordinate system R_{F} obtained from the position of the pliers-shaped element is then identical in the absence or in the presence of operating drapes. As an example, for a cylindrical tracking device comprising three marks on the outer cylindrical lateral wall, the pliers-shaped element comprises three legs connected to a base on which are arranged the marks of the pliers-shaped element. An actuation mechanism enables simultaneously and identically displacing the legs so that each end of a leg bears against a mark of the tracking device, possibly with interposed operating drapes, the cooperation of the three legs with the marks of the tracking device ensuring the attachment of the pliers-shaped element on the tracking device.

FIG. 9A shows a display screen 70 connected to the computer on which the computer can display information to help the surgeon find conical marks 36A, 36B, 36C, 54 covered with the operating drapes. As an example, the computer displays an image 72 representing a portion of pelvis 12 or of any other anatomic element of the patient on which are superposed the positions, represented by circles 74, of conical marks 36A, 36B, 36C, 54 such as determined in reference coordinate system R_{F} on palpation of tracking device 30, 50 in the absence of the operating drapes. The computer also displays an image 76 representing the position of the probe with respect to conical marks 36A, 36B, 36C, 54 in reference coordinate system R_{F}, according to any viewing angle. The surgeon can then more easily find the positions of conical marks 36A, 36B, 36C, 54 when tracking device 30, 50 is covered with operating drapes. It is assumed that location system 19 and thus reference coordinate system R_{F} is substantially fixed. Further, the computer can assign a number to the conical marks determined on palpation of tracking device 30, 50 in the absence of the operating drapes. The next conical mark to be palpated by the surgeon according to the numbering order can then be represented by a symbol 78 different from symbol 74 used for the other conical marks. More detailed information may be displayed in a window 80 inserted in display screen 70.

FIG. 9B illustrates an example of information displayed on window 80. A first symbol 82, for example, a circle, placed at the center of window 80, corresponds to the position of the next conical mark to be palpated by the surgeon according to the numbering order. A second symbol 84, for example, a cross, corresponds to the position of the projection of the center of the end of the probe on a specific plane, for example, the plane perpendicular to the axis of the conical mark associated with first symbol 82 and containing the top of the conical mark. Graduated axes 85, 86 enable estimating the distance in the plane between the probe end and the conical mark to be palpated. The distance separating a cursor 87 from a reference bar 88 is representative of the distance separating the probe end and the conical mark to be palpated along the direction perpendicular to the previously-defined plane. The surgeon can then more easily find the next conical mark to be palpated.

After having found first conical mark 36A, 36B, 36C, 54, the computer may apply a translation to the symbols displayed on display screen 70 corresponding to the other conical marks 36, 54 not yet palpated, equal to the translation between the position of the first conical mark obtained in the absence of operating drapes and the newly-determined position of the first mark in the presence of the operating drapes. This compensates for a possible displacement of location system 19 or of patient 10.

When a number of conical marks greater than three is used, the computer can implement conventional point-aligning methods (for example, according to a lesser square method) to eliminate the points obtained by palpation of conical marks, the positions of which are the most distant from the provided positions or to enable the surgeon to palpate again the conical marks corresponding to the points considered as incorrect.

According to a variation of the present invention, the position of the characteristic anatomic points are obtained with a local ultrasound scan probe which transmits two-dimensional images to the computer and the position and the orientation of which are constantly measured in reference coordinate system R_{F} by means of a rigid body added to the probe and which is similar to the rigid body shown in FIG. 2. The images can be displayed on a screen. The positions of the characteristic anatomic points are, for example, obtained, as follows:

the surgeon visually analyzes the two-dimensional images provided by the ultrasound scan probe and designates points by means of a mouse or of a touch-sensitive display unit;

the computer analyzes the two-dimensional images and automatically locates points having remarkable properties; or

the computer determines from the two-dimensional images a set of points in a three-dimensional space and deforms a three-dimensional pelvis model from the set of determined points to obtain a three-dimensional surface representative of the pelvis of patient 10. The positions of the characteristic anatomic points can then be obtained by searching points of the three-dimensional surface having specific geometric properties or simply resulting from the deformation of the corresponding anatomic points on the deformed model.

According to another variation of the present invention, the characteristic anatomic points of patient 10 are clouds of points that may form portions of curved lines or of surfaces. These points, these lines, and these surfaces may be aligned with three-dimensional images of the patient of scanner or magnetic resonance type according to conventional alignment methods. By combination, the position of these three-dimensional images will be obtained in the coordinate system linked to rigid body 20 once the fields have been arranged.

The present invention has many advantages:

First, it enables easily palpating anatomic points or surfaces on a patient in the absence of the sterile fields, and finding these points or surfaces in a reference frame associated with a rigid body attached to the patient once the sterile fields have been arranged, even if the patient has moved between the two phases.

Second, it enables determining the position of a patient's anatomic element, which position is used in a computer-assisted surgical operation, while limiting risks of additional infection of the patient.

Third, it enables determining the position of the anatomic element even if the patient is covered with operating drapes.

Fourth, the tracking device according to the present invention is arranged on the patient at the level of a region against which the device for maintaining the patient in lateral decubitus position generally bears so that the device according to the present invention does not disturb the surgeon's motions during the operation.

Of course, the present invention is likely to have various alterations and modifications which will readily occur to those skilled in the art. In particular, the present invention has been described in for a computer-assisted arthroplasty of the hip. It should be clear that the present invention can find an application for any computer-assisted operation in which the position of an anatomic element of the patient must be continuously determined even if the patient is covered with operating drapes. As an example, the present invention may apply to a skull surgery operation in which anatomic points or surfaces can be located on the patient's skin before arranging operating drapes and in which the method and the device according to the present invention are used to find these positions after arranging the operating drapes, and thus compensate for a possible motion of the patient between the two phases.

1-13. (canceled)

14. A tracking device (30) in a computer-assisted surgical operation, comprising at least three non-aligned marks (36A, 36B, 36C) likely to be palpated through at least one operating drape (44) and defining first guiding points (C1, C2, C3) in the absence of said at least one operating drape and second guiding points (C1′, C2′, C3′), distinct from the first guiding points, in the presence of the operating drape, the device comprising a mechanical part (30), the three marks (36A, 36B, 36C) being arranged on the mechanical part so that the first guiding points and the second guiding points are coplanar.

15. The device of claim 14, wherein the mechanical part (30) comprises a lateral wall (41A, 41B, 41C), the three marks (36A, 36B, 36C) being arranged on the lateral wall so that the first guiding points and the second guiding points are coplanar.

16. The device of claim 14, wherein at least one mark (36A, 36B, 36C, 54) is a conical recess surrounded with a protruding ring-shaped edge (40A, 40B, 40C, 58).

17. The device of claim 14, comprising a cylindrical block, the three marks being arranged on the outer cylindrical wall of the block.

18. The device of claim 14, comprising a parallelepipedal block (34), the three marks (36A, 36B, 36C) being arranged on three distinct surfaces (41A, 41B, 41C) of the block.

19. The device of claim 18, wherein two marks (36A, 36B) are arranged on two parallel surfaces (41A, 41B), systemically with respect to a plane equidistant to the two parallel surfaces, the third mark (36C) being arranged on a surface (36C) perpendicular to the two parallel surfaces, doser to one of the parallel surfaces.

20. The device of claim 14, comprising an intermediary element to be attached to the mechanical part in the absence of the operating drape or in the presence of the operating drape interposed between the mechanical part and the intermediary element, the intermediary element comprising legs adapted to cooperate with the marks of the mechanical part to ensure the attachment of the pliers-shaped element to the mechanical part.

21. The device of claim 14, wherein the intermediary element comprises additional marks to be palpated, the additional marks of the intermediary element being at the same relative positions with respect to the mechanical part when the intermediary element is attached to the mechanical part in the presence or in the absence of the operating drape.

22. The device of claim 14, wherein the intermediary element comprises an actuation mechanism adapted to simultaneously and identically displace the legs.

23. A method for determining the position of an anatomic element (12) of a patient (10) comprising the steps of
arranging a tracking device (30, 50) fixed with respect to the anatomic element;
determining the position of the anatomic element from the positions of characteristic anatomic points (14, 16, 18) of the patient, the position of the tracking device and the relative position of the anatomic element with respect to the tracking device;
at least partially covering the tracking device with at least one operating drape (44); and
determining the position of the tracking device at least partially covered with said at least one operating drape and deducing therefrom the position of the anatomic element

24. The method of claim 23, comprising the steps of:
arranging the tracking device, wherein the tracking device comprises an intermediary part removably attached to a mechanical part that is fixed with respect to the anatomic element;
determining the relative position of the anatomic element with respect to the intermediary part;
detaching the intermediary part from the mechanical part;
at least partially covering the mechanical part with at least one operating drape;
reattaching the intermediary element to the mechanical part overtop the at least one operating drape; and
determining the position of the intermediary part and deducing therefrom the position of the anatomic element.

25. The method of claim 23, wherein the tracking device (30, 50) is fixed with respect to the pelvis (12) of the patent.

26. The method of claim 23, wherein the positions of characteristic anatomic points (14, 16, 18) of the patient are obtained by palpation of at least three anatomic points (14, 16, 18) of the patient (10).

27. The method of claim 23, wherein the three anatomic points (14, 16) include at least an anatomic point among the group comprising the two anteroposterior iliac spines (14, 16) and the pubic symphysis (18).

28. The method of claim 23, wherein the positions of characteristic anatomic points (14, 16, 18) of the patient are obtained from local ultrasound scan images of the patient (10).

29. The method of claim 23, wherein the positions of the tracking device (30, 50) are obtained by palpation of marks (36A, 36B, 36C, 54) of the tracking device (30, 50).

30. The method of claim 29, wherein the marks (36A, 36B, 36C, 54) of the tracking device (30, 50) are palpated to determine the position of the tracking device in the absence of said at least one operating drape (44) by means of a first probe comprising a spherical end (62, 64) having a first radius and the marks (36A, 36B, 36C, 54) of the tracking device (30, 50) are palpated to determine the position of the tracking device at least partially covered with said at least one drape napkin by means of a second probe comprising a spherical end (62, 64) having a second radius smaller than the first radius of the assumed thickness of the napkins.

31. The method of claim 23, wherein the marks (36A, 36B, 36C, 54) are conical recesses.

32. The method of claim 29, wherein the positions of the marks (36A, 36B, 36C, 54) of the tracking device (30, 50) determined in the absence of said at least one operating drape (44) are displayed on a display screen (70) to ease the palpation of the marks (36A, 36B, 36C, 54) of the tracking device (30, 50) to determine the position of the tracking device at least partially covered with said at least one operating drape.

33. The method of claim 32, wherein the displayed positions of the marks (36A, 36B, 36C, 54) of the tracking device (30, 50) determined in the absence of said at least one operating drape (44) are corrected based on the determined position of a first mark (36A, 36B, 36C, 54) of the tracking device (30, 50) at least partially covered with said at least one operating drape.
